(19) United States
(12) Patent Application Publication
(10) Pub. No.: US 2015/0149199 A1
(43) Pub. Date: May 28, 2015

(54) INTEGRATED POINT OF CARE AND HOME MEDICATION DELIVERY METHOD

(71) Applicants: Kevin Thomas FOLEY, Germantown, TN (US); Kyle Steven Gabrick, Germantown, TN (US)

(72) Inventors: Kevin Thomas FOLEY, Germantown, TN (US); Kyle Steven Gabrick, Germantown, TN (US)

(21) Appl. No.: 14/551,756

(22) Filed: Nov. 24, 2014

Related U.S. Application Data
(60) Provisional application No. 61/907,718, filed on Nov. 22, 2013.

(51) Int. Cl. G06F 19/00; G06Q 10/08; G06Q 20/34

(52) U.S. Cl. CPC G06F 19/3462; G06F 19/328; G06Q 20/34; G06Q 10/083
USPC 705/2

(57) ABSTRACT

A method for supplying a prescribed medication to a patient. A medication prescription order and information on a patient is sent by an authorized person in a medical office to a PPC for verification of insurance and billing information. Alternatively, the information may be sent to a PDU in the medical office. Upon verification of the insurance and billing information, the PPC or PDU sends an access code to an authorized person in the medical office. Upon receipt of the access code, the authorized person enters it in the PDU to obtain a temporary supply of the prescribed medication for the patient. The PDU sends an order for the prescribed medication to the PPC. The PPC fills the order and ships the prescribed medication to a patient's location to arrive before the patient has used all of the temporary supply of the prescribed medication.

This application claims the priority of Provisional Patent Application No. 61/907,718 filed on Nov. 22, 2013.

### BACKGROUND OF THE INVENTION

1. Field of the Invention

The present invention relates to a delivery method for medications and, more particularly, to such a method that integrates point of care medication dispensing to home delivery of medications.

2. Description of the Background Art

In the present environment, doctors and authorized staff order medications for patients and send the patients to pharmacies to pick up the medications. First, it is inconvenient and costs time and money for a patient to visit a pharmacy to pick up a medication. Second, the patient may not get the medication as quickly as needed. Third, the patient must return to the pharmacy for refills. This method by which patients fill their prescriptions is highly inefficient and contributes significantly to medical non-compliance.

Currently, a patient must opt to have a prescription filled at a local retail pharmacy, have a medication dispensed from a vending machine or the like located in a clinic, or have the medication provided by a mail order pharmacy. All of these models have limitations for streamlined integration.

Accordingly, a need has arisen for a new and improved method of dispensing prescription medications to patients. The method of the present invention meets this need.

### BRIEF SUMMARY OF THE INVENTION

In accordance with one method of the present invention, an authorized person such as a doctor, nurse or medical assistant in a medical office such as a doctor's office or clinic, sends a prescription order and patient demographics by telephone, electronically or the like to a processing unit or a database at a Pharmacy Processing Center (PPC) for insurance and billing information verification on the patient. The PPC is a site at which the billing and demographic information is processed for each prescription, and may be one central building or pharmacy or a network of existing pharmacies. It is important that the PPC be operable to process a written, faxed or electronically received prescription and, when authorized, can dispense a medication that can be shipped directly, or through a third party shipping and handling carrier to the patient.

Once the PPC verifies insurance and billing information, an access code or information is sent electronically to the medical office and to a Prescription Dispensing Unit (PDU) at the medical office. An authorized person at the medical office then obtains payment from the patient for the prescription and enters the access code in the PDU to obtain a limited supply of the prescribed medication for immediate use by the patient before the patient receives the full prescription of medication from the PPC.

The PDU preferably is an automated vending machine or location which is constructed to store and dispense a limited supply of prescription medicines, transmit billing and demographic information on patients; maintain a live inventory and order prefills, generate report drug utilization reviews to both a medical office and the PPC, as well as being integrated to the electronic medical records system of the medical office.

Upon verification of payment by the patient by credit card or the like, the PDU sends electronically the prescription order and shipping information for the patient to the PPC and the PPC thereafter fills the order and ships the prescribed medication to a location designated by the patient. The PPC may also send a claim electronically to an insurance carrier for the filling of the prescription order. The PPC also pays a fee to the medical office for the prescription dispensed, for providing the patient counsel on adverse effects of the medication, as well as verification that the patient received the correct medication.

In a modified method of the present invention, the prescription order and patient demographics may be sent by the authorized person directly to the PDU rather than the PPC for insurance and billing information verification. The PDU would then register the patient, and collect payment for the prescription. The PDU would also provide the authorized person at the medical office with an appropriate access code or information so that the authorized person could dispense the temporary amount of prescription medication to the patient for use before the patient receives the prescription order from the PPC.

Another benefit is it streamlines the process by which the medical practice stocks prescription units in anticipation of dispensing. Instead of keeping precise quantities required for full therapeutic regimens. They can now stock limited supplies and the differential between the limited supply and the full regimen is made up with mail order. Thus, The medical practice needs only stock one limited supply where previous in clinic dispensing apparatuses must have kept varying quantities of medication which greatly simplifies the formulatory of medication required by a medical clinic.

From the foregoing description, it is apparent that the method of the present invention is very advantageous in that the patient can immediately receive an initial or temporary supply of a prescribed medication at the medical office and can then receive the full prescription by mail or otherwise from the PPC without having to visit a pharmacy to have the prescription filled. Also, the medical office receives electronically a confirmation that the patient has received the medication from the PPC and also receives payment from the PPC for the service rendered in ordering the prescription.

### BRIEF DESCRIPTION OF THE DRAWING

FIG. 1 is a flow diagram illustrating the steps in the method of the present invention.

### DETAILED DESCRIPTION OF THE INVENTION

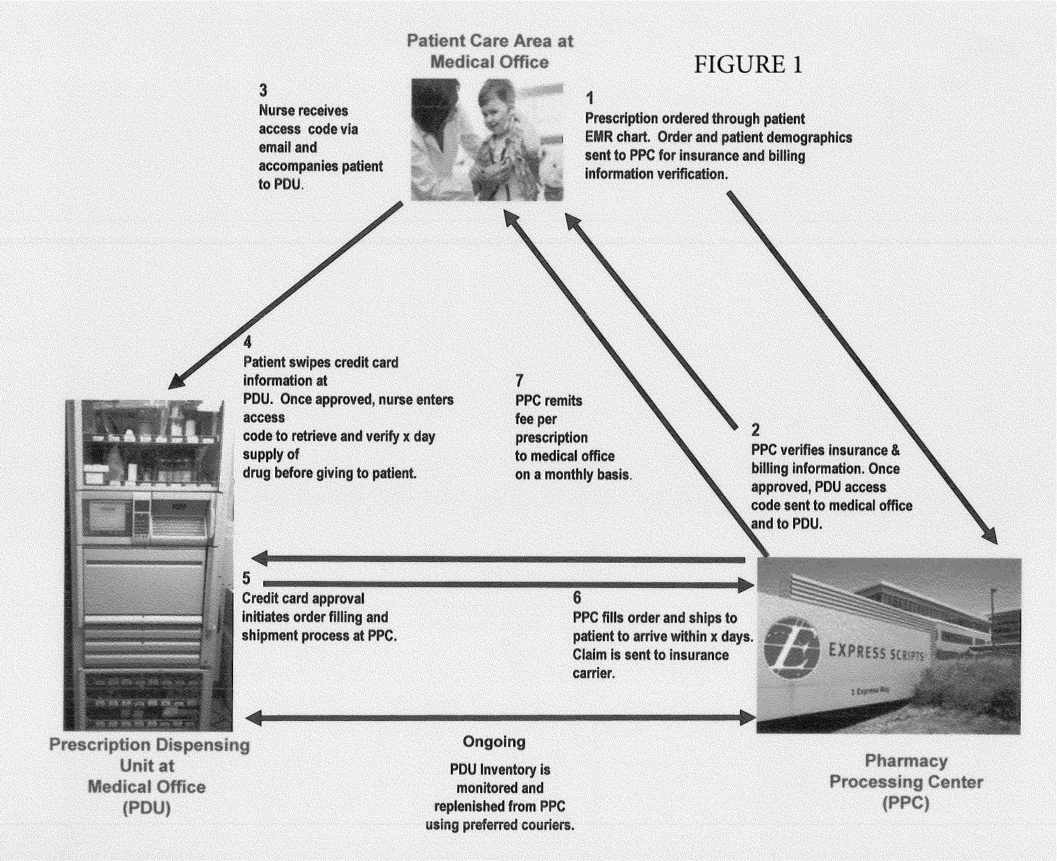

Referring to FIG. 1, after concluding that a patient requires medication, an authorized person at a medical office, in accordance with one method of the present invention, sends the prescription information and patient demographics by telephone or electronically to a PPC for insurance and billing information verification according to step 1.

Thereafter, the PPC verifies insurance and billing information and, once approved, sends an access code to the medical office and to a PDU located at the medical office in accordance with step 2.

An authorized person at the medical office receives the access code from the PPC and visits the PDU at the medical office in accordance with step 3 of the present method.

At the PDU, the authorized person enters the access code to receive a temporary supply of the prescribed medication for the patient and arranges for payment for the temporary supply by the patient by credit card or the like in accordance with step 4 of the present method.

Once payment is approved and the temporary supply of the medication is provided to the patient, the PDU sends an order and shipping information for the medication prescription to the PPC in accordance with step 5 of the method.

The PPC then fills the order and ships the prescribed medicine to the home of the patient or other location identified by the patient to arrive before the patient uses all of the temporary supply of the medication from the PDU. The PPC also sends a claim for the shipped prescription to the insurance carrier for the patient, in accordance with step 6.

The PPC also sends a payment to the medical office for the prescription order in accordance with step 7.

The inventory of the PDU is monitored and replenished periodically to ensure an adequate supply of medications to be supplied to patients on a temporary basis while they are awaiting their prescriptions from the PPC.

In accordance with a modified method of the present invention, the order and patient demographics may be sent by the authorized person in the medical office directly to the PDU for insurance and billing information verification. The PDU would then be operable to provide the access code to the authorized person so that the temporary supply of the prescribed medication can be obtained from the PDU by the authorized person after receiving payment from the patient. All of the other steps would be the same as the hereinbefore described method.

From the foregoing description, it is apparent that the new and improved method of the present invention results in higher quality patient care, improved patient convenience, improved patient compliance, lower costs to patients and revenue opportunities for medical offices.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A method for integrating the supply of a prescribed medication to a patient in a medical office and the delivery of the medication to a location identified by the patient, wherein:
the information on a medication prescription order for the patient and patient demographics is sent by an authorized person in a medical office to a PPC for verification of insurance and billing information for the patient;
upon verification of the insurance and billing information, the PPC sends an access code or information to an authorized person and to a PDU in the medical office;
upon receipt of the access code or information, the authorized person enters the access code or information in the PDU to obtain a temporary supply of the prescribed medication for the patient and obtains payment from the patient for the temporary supply;
the PDU sends an order for the prescribed medication and shipping information to the PPC; and
the PPC fills the order and ships the prescribed medication to the location identified by the patient to arrive before the patient has used all of the temporary supply of the prescribed medication.

2. The method of claim 1 wherein the PPC sends a payment to the medical office for the medication prescription order.

3. The method of claim 1 wherein the information on the medication prescription order is sent by telephone or electronically by the authorized person to the PPC.

4. The method of claim 1 wherein the access code or information is sent electronically by the PPC to the authorized person and PDU at the medical office.

5. The method of claim 1 wherein the prescribed medication order and shipping information are sent electronically from the PDU to the PPC.

6. The method of claim 1 wherein the PDU is operable to obtain and verify credit card information from the patient.

7. The method of claim 1 wherein the PDU is operable to provide a predetermined supply of the prescribed medication to the patient so that it will last at least until the patient receives the prescribed medication from the PPC.

8. The method of claim 1 wherein the PDU is electronically connected to an electronic medical record system of the medical office.

9. A method for integrating the supply of a prescribed medication to a patient in a medical office and the delivery of the medication to a location identified by the patient, wherein
the information on a medication prescription order for the patient and patient demographics is sent by an authorized person in a medical office to a PDU in the medical office for verification of insurance and billing information for the patient;
upon verification of the insurance and billing information, the PDU sends an access code or information to an authorized person in the medical office,
upon receipt of the access code or information, the authorized person enters the access code or information in the PDU to obtain a temporary supply of the prescribed medication for the patient and obtains payment from the patient for the temporary supply;
the PDU sends an order for the prescribed medication and shipping information to a PPC; and
the PPC fills the order and ships the prescribed medication to the location identified by the patient to arrive before the patient has used all of the temporary supply of the prescribed medication.

10. The method of claim 9 wherein the PPC sends a payment to the medical office for the medication prescription order.

11. The method of claim 9 wherein the information on the medication prescription order is sent by telephone or electronically by the authorized person to the PDU.

12. The method of claim 9 wherein the prescribed medication order and shipping information are sent electronically from the PDU to the PPC.

13. The method of claim 9 wherein the PDU is operable to obtain and verify credit card information from the patient.

14. The method of claim 9 wherein the PDU is operable to provide a predetermined supply of the prescribed medication to the patient so that it will last at least until the patient receives the prescribed medication from the PPC.

15. The method of claim 9 wherein the PDU is electronically connected to an electronic medical record system of the medical office.
